(19) United States
(12) Patent Application Publication
(10) Pub. No.: US 2023/0414571 A1
(43) Pub. Date: Dec. 28, 2023

(54) APPLICATION OF SMALL-MOLECULE COMPOUND IN PREPARATION OF ANTITUMOR DRUG

(71) Applicant: SOOCHOW UNIVERSITY, Jiangsu (CN)

(72) Inventors: Jian LIU, Jiangsu (CN); Rui SHI, Jiangsu (CN); Peichen PAN, Jiangsu (CN); Rui LV, Jiangsu (CN); Zhenhui KANG, Jiangsu (CN); Tingjun HOU, Jiangsu (CN)

(73) Assignee: SOOCHOW UNIVERSITY, Jiangsu (CN)

(21) Appl. No.: 18/466,829

(22) Filed: Sep. 14, 2023

Related U.S. Application Data
(63) Continuation of application No. PCT/CN2021/101767, filed on Jun. 23, 2021.

(30) Foreign Application Priority Data
Apr. 21, 2021 (CN) 202110429488.X

(51) Int. Cl. A61K 31/417; A61K 31/381; A61K 31/4402; A61K 31/165; A61K 31/166; A61K 31/4409; A61K 31/4406; A61K 31/137; A61K 31/505; A61K 31/496; A61P 35/00; H01J 49/16

(52) U.S. Cl. CPC A61K 31/417; A61K 31/381; A61K 31/4402; A61K 31/165; A61K 31/166; A61K 31/4409; A61K 31/4406; A61K 31/137; A61K 31/505; A61K 31/496; A61P 35/00; H01J 49/164

(57) ABSTRACT

The present invention provides an application of a small-molecule compound in preparation of an antitumor drug. A series of small-molecule drugs with high brain tumor growth activity inhibitory properties are obtained, are docked to pharmacodynamic analysis of the small-molecule drugs, and do not need to be transferred to other testing platforms.

### CROSS-REFERENCE TO RELATED APPLICATION

This application is a continuation of international application of PCT application serial no. PCT/CN2021/101767, filed on Jun. 23, 2021, which claims the priority benefit of China application no. 202110429488.X, filed on Apr. 21, 2021. The entirety of each of the above-mentioned patent applications is hereby incorporated by reference herein and made a part of this specification.

### BACKGROUND OF THE INVENTION

### 1. Technical Field

The present invention belongs to the technical field of analysis and testing, and particularly relates to an application of a small-molecule compound in preparation of an antitumor drug.

### 2. Background Art

The matrix-assisted laser desorption ionization mass spectrometry is a soft ionization technology, and has a great success in rapidly analyzing biomacromolecules (nucleic acids, proteins, polypeptides, etc.) and polymers. However, a conventional organic matrix commonly used in the matrix-assisted laser desorption ionization mass spectrometry (MALDI-MS) is likely to generate very high background noise when a mass-to-charge ratio is lower than 700, which disturbs signals for mass spectrometry analysis of small molecules. Moreover, the organic matrix tends to form randomly distributed crystals of different sizes (tens of microns in magnitude), thereby reducing reproducibility of the signals, and thus seriously hindering development of the MALDI in small molecule analysis and testing, and application.

In reports of the prior art, inorganic nanomaterials or inorganic nanostructure surfaces have been applied as suitable matrices in the MALDI to replace organic matrices, including silicon, alloys, metal oxides, carbon nanomaterials, etc. These inorganic nanomaterials or inorganic nanostructure surfaces overcome the background interference of traditional organic matrices in a low molecular weight region, but are difficult for sensitive testing and accurate imaging of small molecules in vivo, and cannot be used for rapid screening of new drugs.

Although these materials are greatly improved in analysis of small molecules, the obtained mass spectrometry is not high in sensitivity and poor in repeatability, such that there is a big defect in an analysis of natural products, blood and biological samples, and an application of direct tissue imaging.

The process from computer screening to obtaining of target molecules is fast, but actual experimental conditions such as reagents are lacked in a computer-simulated drug environment, which is still the defect of the method. An additional artificial substrate modification or coupling enzymes are required in a common fluorescence or ultraviolet absorption method, which is likely to cause the problem of interfering candidate drug signals. Existing optimal liquid chromatography-tandem mass spectrometry (LC-MS) systems equipped with ultra-fast gradient conventional short columns are still limited in speed, and only 768 parts of blood samples are analyzed within 3.75 hours (excluding a large amount of pre-processing time).

### SUMMARY OF THE INVENTION

In view of this, an objective of the present invention is to provide an application of a small-molecule compound in preparation of an antitumor drug. The method is quickly effective and highly accurate.

The present invention provides a screening method for a hexokinase 2 inhibitor, and the method includes the following steps:
mixing a candidate inhibitor and a buffer solution, performing incubation, and terminating a reaction to obtain a post-reaction solution, where the buffer solution includes hexokinase 2 and glucose;
uniformly mixing the post-reaction solution with an equal volume of glucose-1-¹³C to obtain an object to be analyzed; and
dropwise adding the object to be analyzed to a surface of a graphite structure type nanomaterial matrix, performing drying, then, performing matrix-assisted laser desorption ionization mass spectrometry (MALDI-MS) testing, and performing screening to obtain the hexokinase 2 inhibitor.

In the present invention, the screening conditions are as follows:
a concentration of the candidate inhibitor aqueous solution is 20 μmol/L, an inhibition rate of hexokinase 2 activity is ≥25%, and

an inhibition rate=(1−B/A)×100%, where
A is: a glucose concentration in a pre-reaction solution without the inhibitor-a glucose concentration in a post-reaction solution without the inhibitor,
B is: a glucose concentration in a pre-reaction solution with the inhibitor-a glucose concentration in a post-reaction solution with the inhibitor, and

a glucose concentration in the reaction solution=(mass spectrum signal strength of [glucose+Na]⁺/mass spectrum signal strength of [glucose-1-¹³C+Na]⁺)×known concentration of glucose-1-¹³C.

In the present invention, a volume ratio of the candidate inhibitor to the buffer solution is 1:1,
a mass ratio of the hexokinase 2 in the buffer solution to glucose substance is (0.245-0.255) g: 0.5 mmol, and
a concentration of the candidate inhibitor is 1 μmol/L-1000 μmol/L.

In the present invention, an incubation temperature is 35-40° C., and incubation time is 55-65 min.

In the present invention, a reflection positive and negative ion mode is employed in the MALDI-MS testing.

Experimental Parameters for MALDI-MS Testing:
Nd of 355 nm: YAG laser has laser energy of 30%, corresponding to 57 μJ per pulse, a laser pulse duration is 3 ns, and a laser spot size is 50-100 μm; and
each sample is repeatedly tested four times, and 3000 laser spots are accumulated in each mass spectrum.

In the present invention, the graphite structure type nanomaterial matrix (namely graphite dots, GDs) mainly contains three functional groups, namely a hydroxyl group (—OH), a carbonyl group (C═O) and an epoxy group (C—O—C). Percentages of the surface functional groups of the GDs: C—OH, 26%; C═O, 13%; and C—O—C, 1%. The GDs have good dispersibility, and have a uniform particle size distribution of about 5-6 nm, a uniform height (approximately 6 nm in height), which indicates that the GDs are approximately of one cubic block, a honeycomb graphite structure, and a standard hexagonal crystal structure. Strong ultraviolet absorption exists at the positions of 337 nm and 355 nm, which covers a laser wavelength most widely used by the MALDI-MS.

In the present invention, the candidate inhibitor is obtained through the following method:
performing screening by using a LigPrep module of Schrödinger to obtain a screened compound;
in a simulation environment in which a protonation state parameter is pH=7.0+/−2.0, a scale factor of the Van der Waals radius is set to be 1.0, and a maximum partial atomic charge is set to be 0.25, firstly, scoring and sorting binding affinity of the preliminary target compound and HK2 by using a Glide-SP docking mode, then scoring the 50000 compounds ranking top by using a Glide XP docking mode, next, predicting the absorption, distribution, metabolism, excretion, toxicity (ADMET) properties of the selected compounds by using an ACD/ADME software package, and filtering the 1000 compounds ranking top in Glide XP docking to remove those compounds which do not conform to the following rules: (1) log P/log D (pH=7.0)<5.5, (2) violating the Lipinski Rule of Five and being <2, (3) violating a drug similarity rule of Opera and being <3, and (4) functional groups having no toxicity, reactivity or other bad portions defined by the REOS rule; and
finally, clustering the remaining molecules by using a Find Diversity Molecule module in Discovery Studio 2.5 according to a Tanimoto distance calculated by FCFP_4 fingerprints, thereby obtaining the candidate inhibitor.

The method provided by the present invention may be applied in the fields of mass spectrometry assay, mass spectrometry imaging, proteomics, metabonomics, drug research and development, and drug analysis and application.

In the present invention, the graphite structure type nanomaterial matrix is taken as a novel matrix in the above-mentioned screening method, and the matrix-assisted laser desorption ionization (MALDI) mass spectrometry technology is combined to identify and measure reactants (or products) involving chemical reactions involving various small molecules so as to screen a small-molecule compound having a specific chemical structure and activity. In the present invention, by using the above technology, absorption and uptake, a blood concentration, an organ distribution, and chemical structure change occurring during these processes of the specific small-molecule compound in a living body experiment are monitored. In the present invention, by using the above technology in combination with tissue morphological characteristics of a three-dimensional space of a living organ, visual testing is performed on a plurality of small molecules distributed therein. In the method, screening is performed to obtain the small-molecule compound for regulating and controlling tumor cell metabolism reprogramming and an antitumor activity, and a derivative thereof.

The hexokinase 2 inhibitor obtained by means of screening through the above screening method is a small-molecule compound, and can be applied to preparation of an antitumor drug.

The present invention further provides an application of a small-molecule compound in preparation of an antitumor drug.

The small-molecule compound has a structure of formula 1 to formula 45:

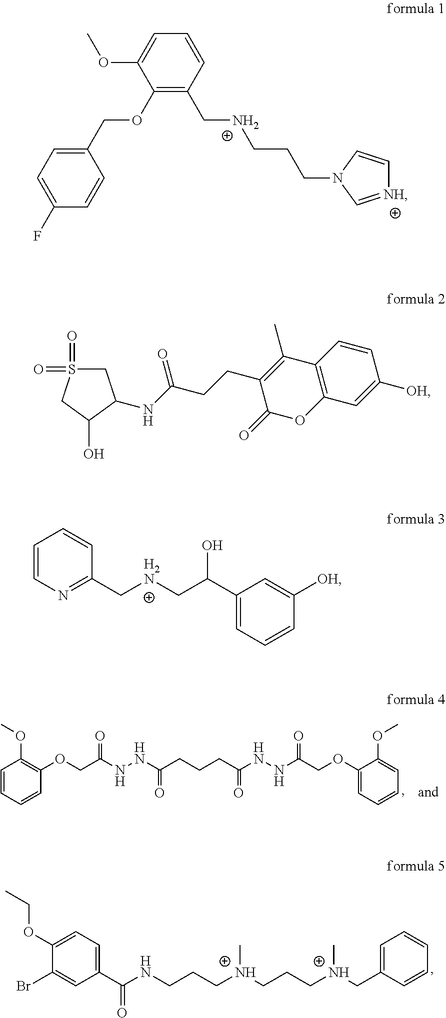

where
compositions of the structure of the above compounds mainly lie in that aromatic groups at both ends are connected by means of a carbon chain, an amide, a methoxy group, and a hydroxyl group are contained in the carbon chain, and a halogen substitution, a hydroxyl group, an epoxy group substitution, etc. are on the aromatic groups.

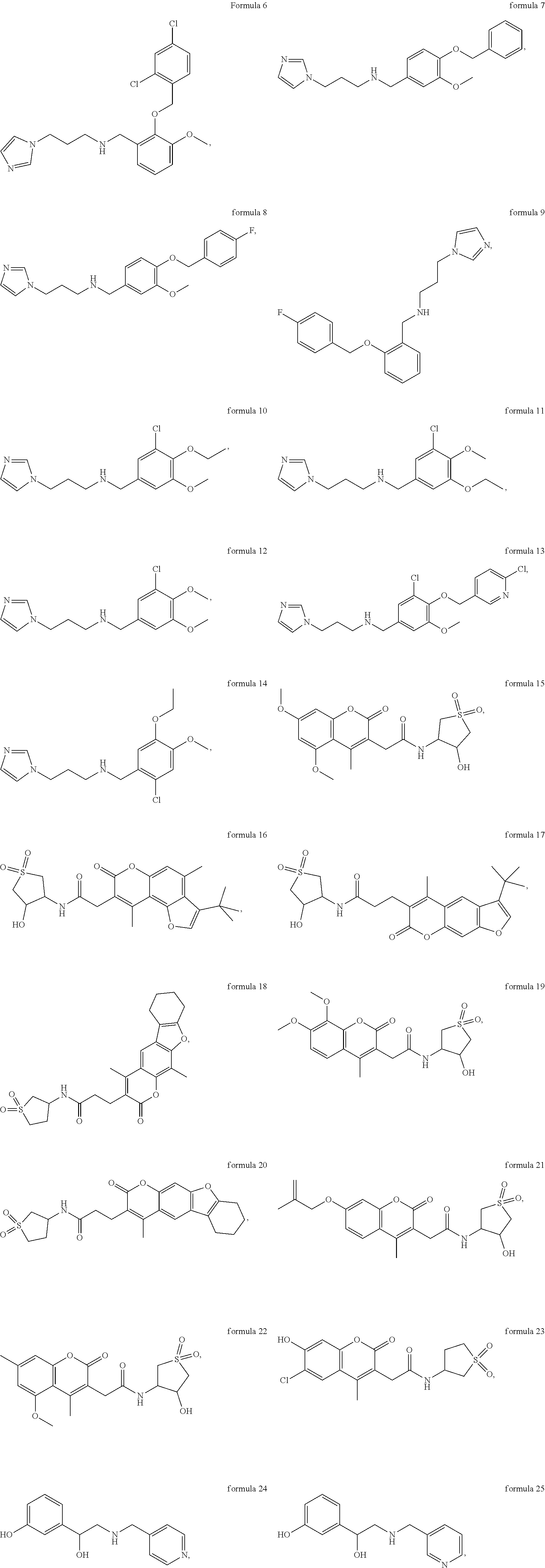

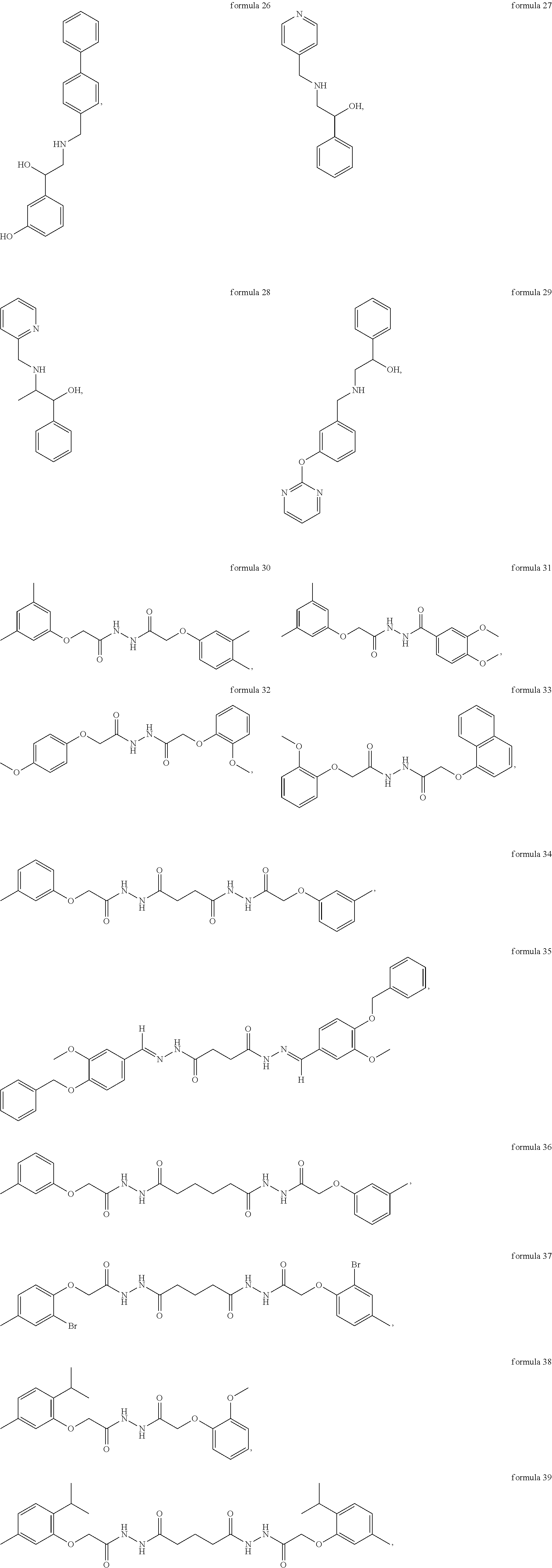

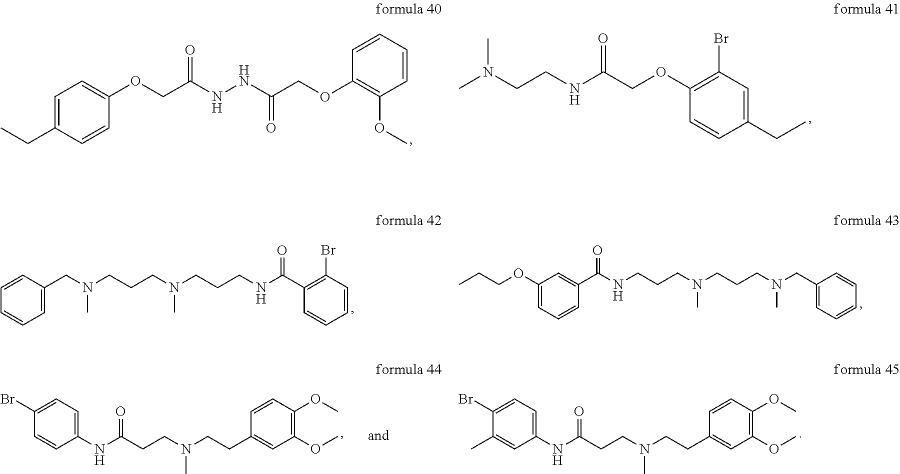 [SMILES: CCc1ccc(OCC(=O)NNC(=O)COc2ccccc2OC)cc1 ; CCc1ccc(OCC(=O)NCCN(C)C)c(Br)c1 ; CN(CCCNC(=O)c1ccccc1Br)CCCN(C)Cc1ccccc1 ; CCCOc1cccc(C(=O)NCCCN(C)CCCN(C)Cc2ccccc2)c1 ; COc1ccc(CCN(C)CCC(=O)Nc2ccc(Br)cc2)cc1OC ; COc1ccc(CCN(C)CCC(=O)Nc2ccc(Br)c(C)c2)cc1OC]

The above-mentioned compounds in formulas 6-14 are all derivatives of the small-molecule inhibitor of formula 1, and are mainly characterized in a six-membered ring side chain substitution on a right side of NH, such as, methyl, benzene ring, and halogen substitutions. The compounds in formulas 15-23 are all derivatives of the small-molecule inhibitor of formula 2, and are mainly characterized in a naphthalene ring side chain substitution on a right side of formamide, such as methoxy and five-membered ring substitutions. The compounds in formulas 24-29 are all derivatives of the small-molecule inhibitor of formula 3, including a benzene ring phenyl substitution on a right side of NH, N shifted on the benzene ring, carbon chain methyl and benzene ring substitution. The compounds in formulas 30-40 are all derivatives of the small-molecule inhibitor of formula 4, including a carbon chain substitution on a right side of NH, a methyl substitution on a benzene ring, etc. The compounds in formulas 41-45 are all derivatives of the small-molecule inhibitors of formula 5, including a benzene ring phenyl substitution on a right side of NH, N shifted on a benzene ring, carbon chain methyl, distal benzene ring substitution, etc.

In the present invention, the small-molecule compound has an inhibitory effect on a key protease in glycolysis abnormality caused by metabolic reprogramming of tumor cells, and is used alone as a pharmaceutical molecule or used in combination with other known drugs to kill the tumor cells.

In the present invention, the small-molecule compound can hinder the capacity of the tumor cell of repairing damaged deoxyribonucleic acid (DNA) during chemotherapy by inhibiting an abnormal metabolic pathway of the tumor cells.

The small-molecule compound can break through restriction of a blood-brain barrier, and is captured and enriched at a tumor site where a drug is usually difficult to reach.

In the present invention, in the case of single administration or continuous multiple administration, quantitative mass spectrometry analysis of drug molecules is performed on a sample with a blood taking amount of less than 10 microliters, such that data acquisition of pharmacokinetics (blood drug concentration-time curve) can be completed by means of a single model animal. The drug refers to the small-molecule compound.

The present invention provides the method for screening the hexokinase 2 inhibitor. The method includes the following steps: mixing the candidate inhibitor and the buffer solution, performing incubation, and terminating the reaction to obtain the post-reaction solution, where the buffer solution includes the hexokinase 2 and the glucose; uniformly mixing the post-reaction solution with the equal volume of glucose-1-¹³C to obtain the object to be analyzed; and dropwise adding the object to be analyzed to the surface of the graphite structure type nanomaterial matrix, performing drying, then, performing the MALDI-MS testing, and performing screening to obtain the hexokinase 2 inhibitor. According to the method, screening and testing are performed at an ultra-rapid speed (1836 samples are analyzed within 5.1 hours) by taking the graphite structure type nanomaterial as the matrix in combination with the MALDI-MS testing. A series of small-molecule drugs with high brain tumor growth activity inhibitory properties are obtained, are docked to pharmacodynamic analysis of the small-molecule drugs, and do not need to be transferred to other testing platforms.

### BRIEF DESCRIPTION OF THE DRAWINGS

FIG. 1 is a schematic diagram of a GLMSD platform for high-throughput screening of a hexokinase 2 (HK2) candidate inhibitor;

FIG. 2 shows inhibition rates of 38 candidate inhibitors against HK2 at four different concentrations;

In FIG. 3, a is a structural formula of compound 8, b shows inhibition curves of HK2 from compound 8 by means of a colorimetric kit and GLMSD, and c shows original data of an HK2 activity of compound 8 at four concentrations tested by a GLMSD platform;

In FIG. 4, a is a structural formula of compound 11, b shows inhibition curves of HK2 from compound 11 by means of a colorimetric kit and GLMSD, and c shows original data of an HK2 activity of compound 11 at four concentrations tested by a GLMSD platform;

In FIG. 5, a is a structural formula of compound 13, b shows inhibition curves of HK2 from compound 13 by means of a colorimetric kit and GLMSD, and c shows original data of an HK2 activity of compound 11 at four concentrations tested by a GLMSD platform;

In FIG. 6, a is a structural formula of compound 21, b shows inhibition curves of HK2 from compound 21 by means of a colorimetric kit and GLMSD, and c shows original data of an HK2 activity of compound 21 at four concentrations tested by a GLMSD platform;

In FIG. 7, a is a structural formula of compound 27, b shows inhibition curves of HK2 from compound 27 by means of a colorimetric kit and GLMSD, and c shows original data of an HK2 activity of compound 11 at four concentrations tested by a GLMSD platform;

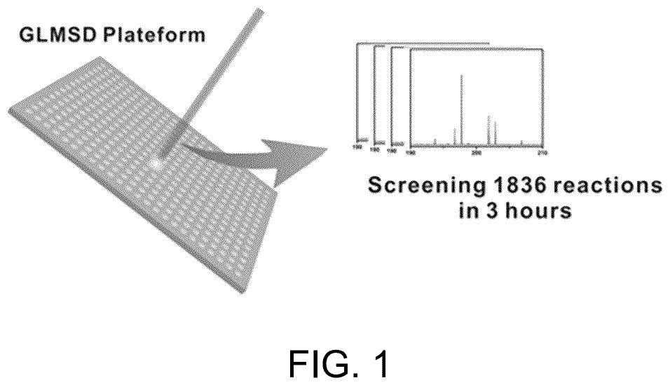

FIG. 8-1 shows pharmacokinetic data of Trimetazidine (TMZ);

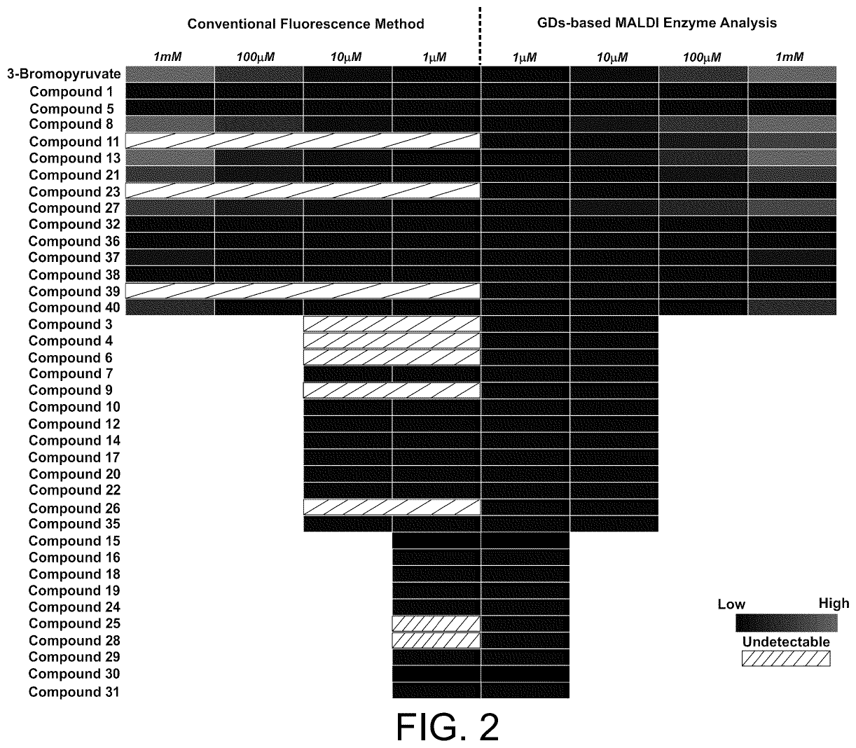

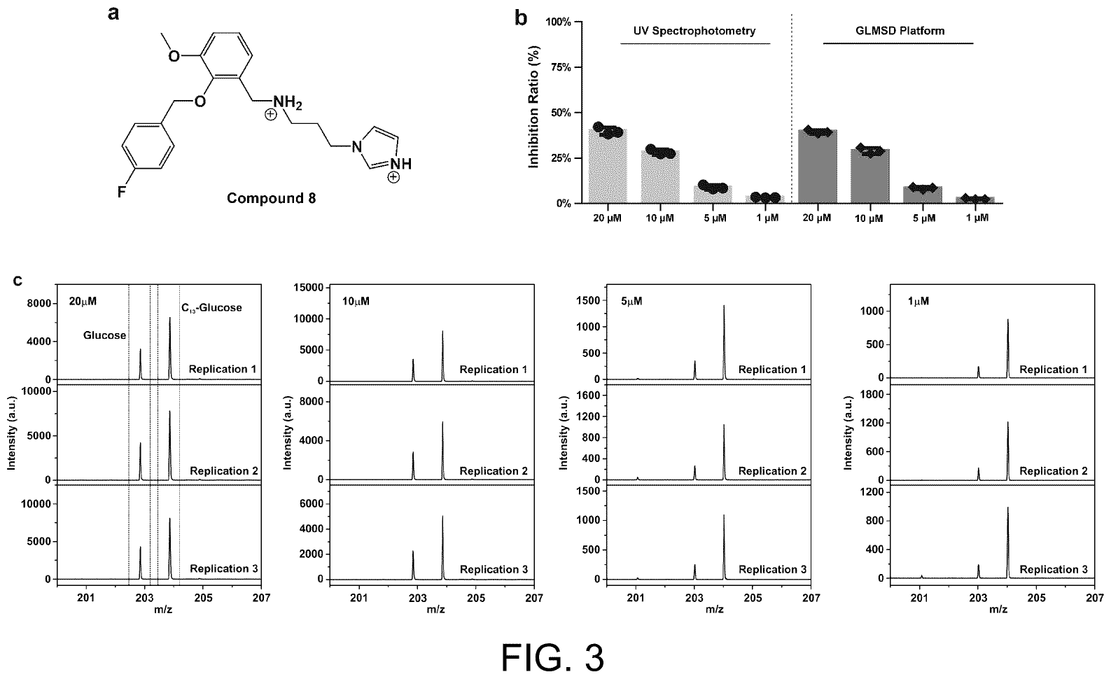

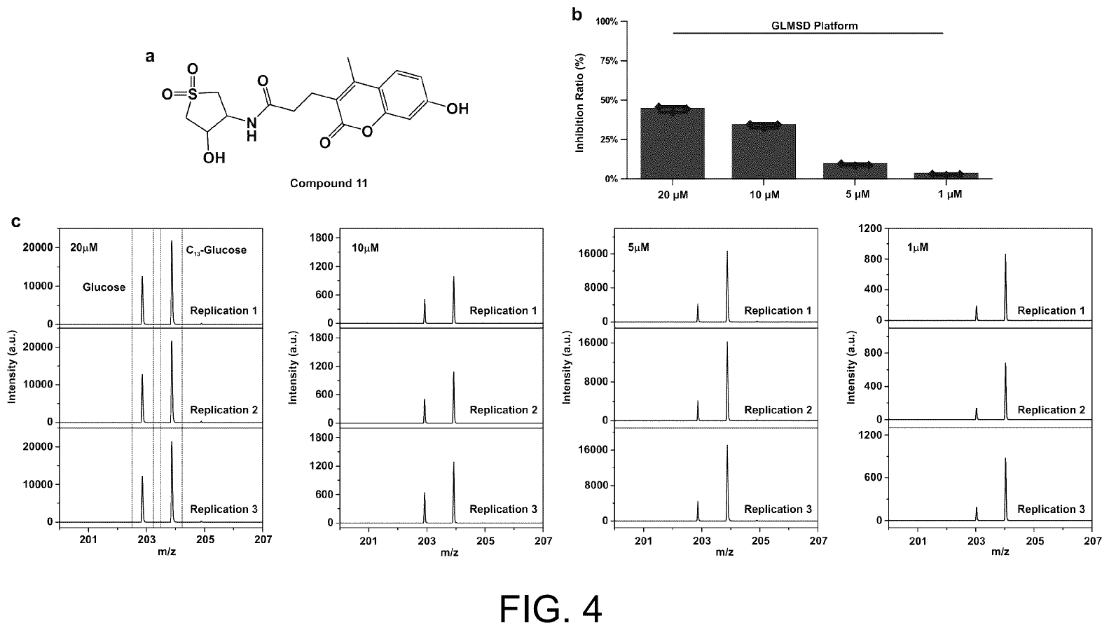

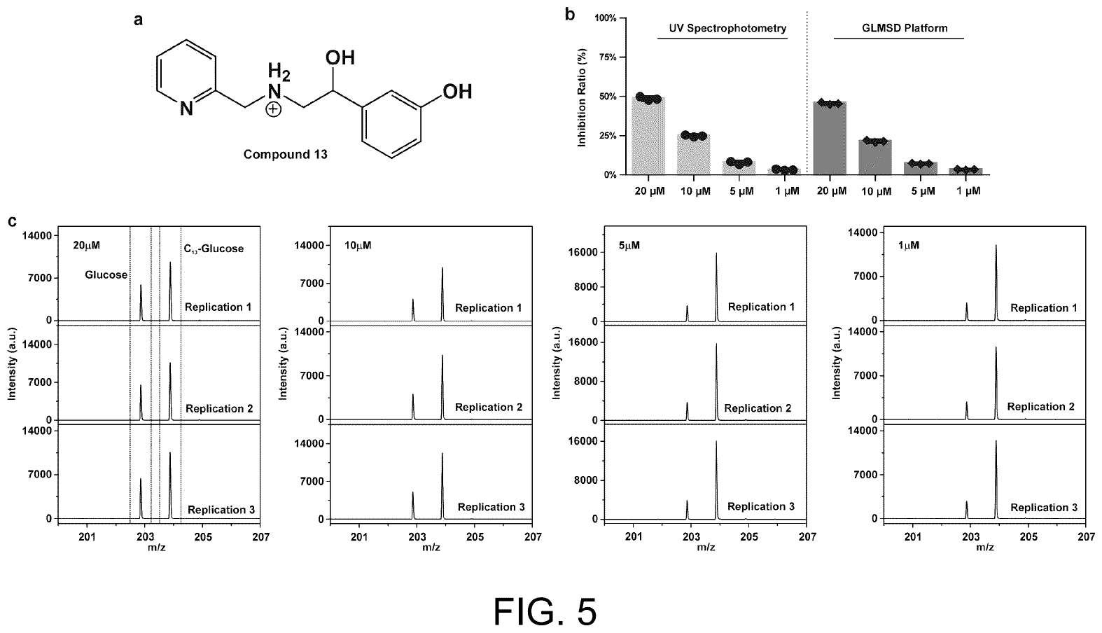

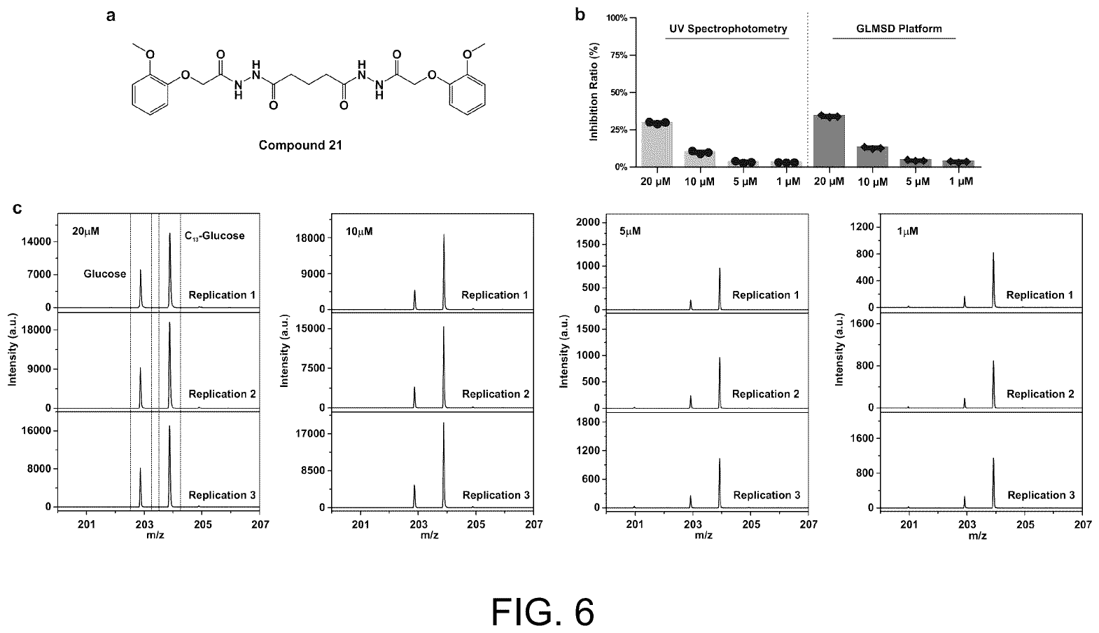

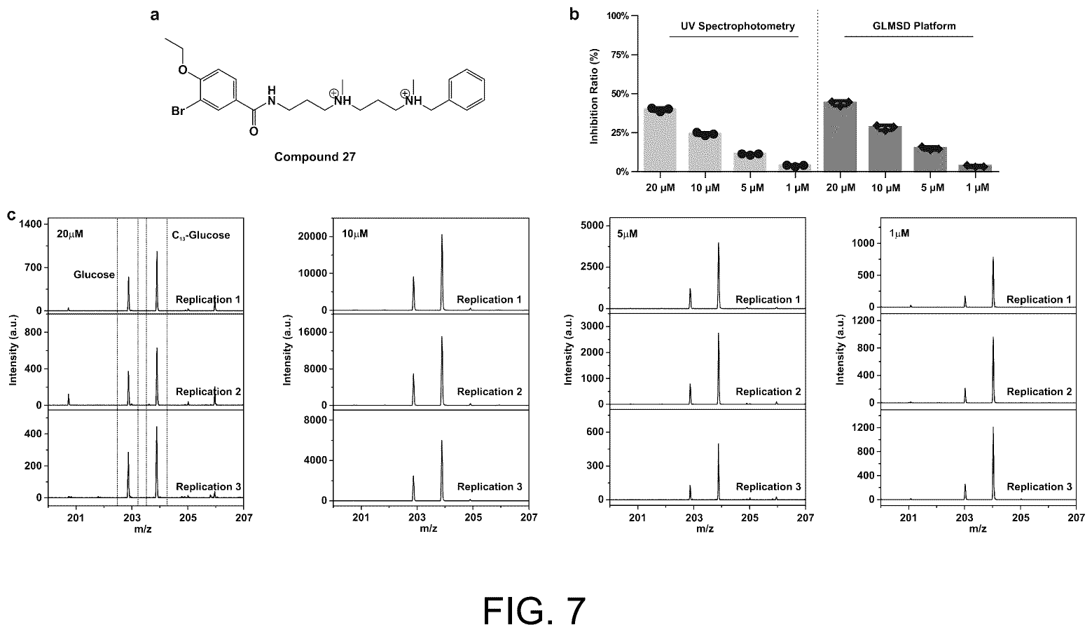

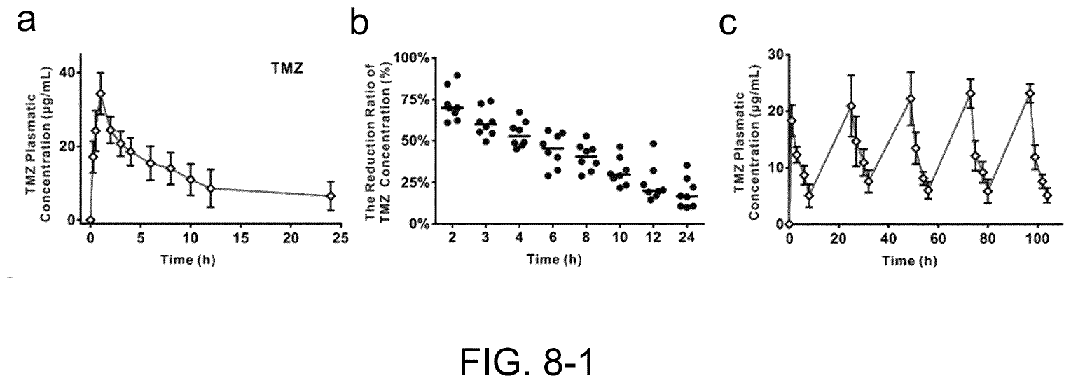

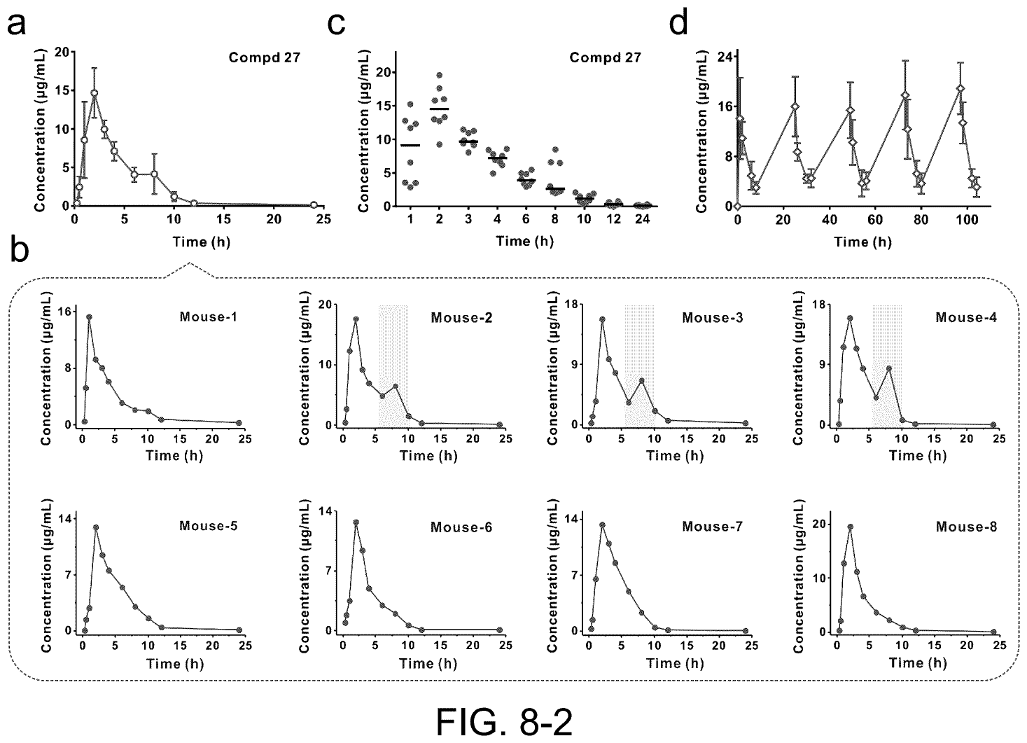

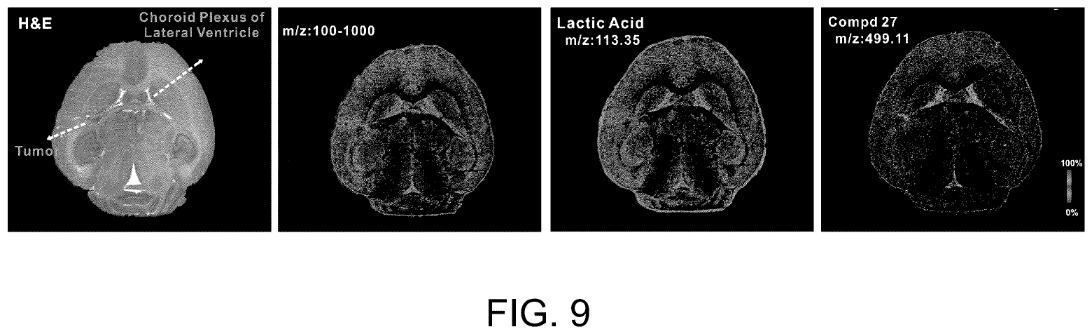

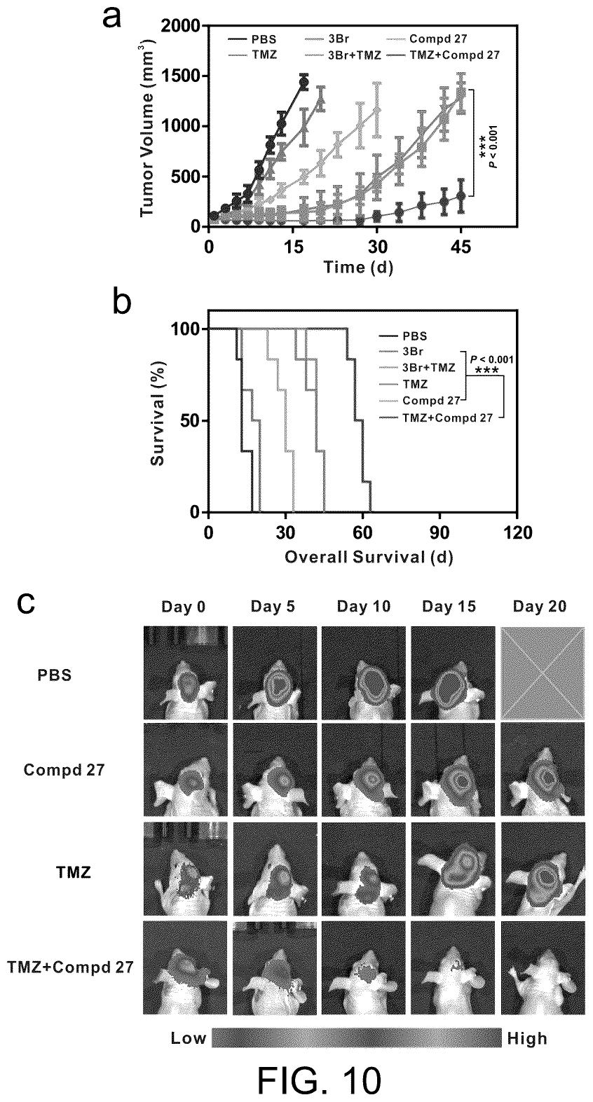

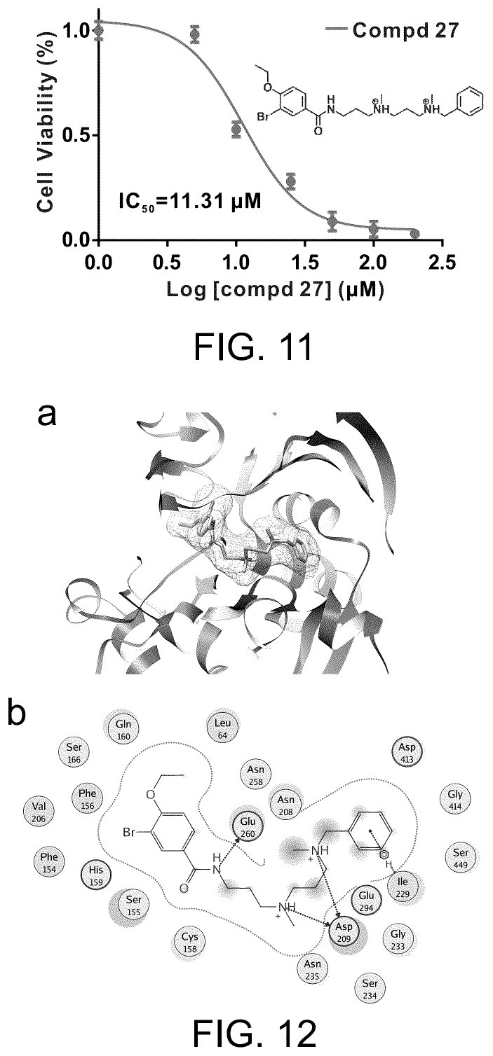

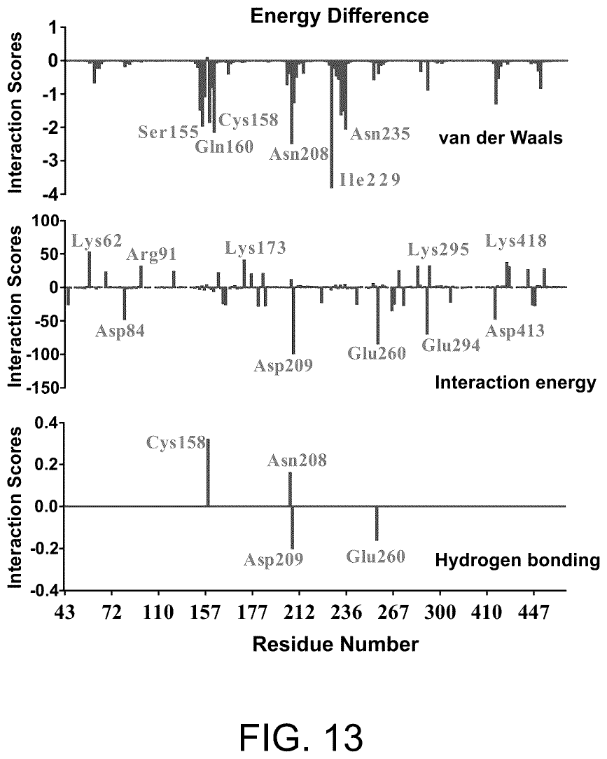

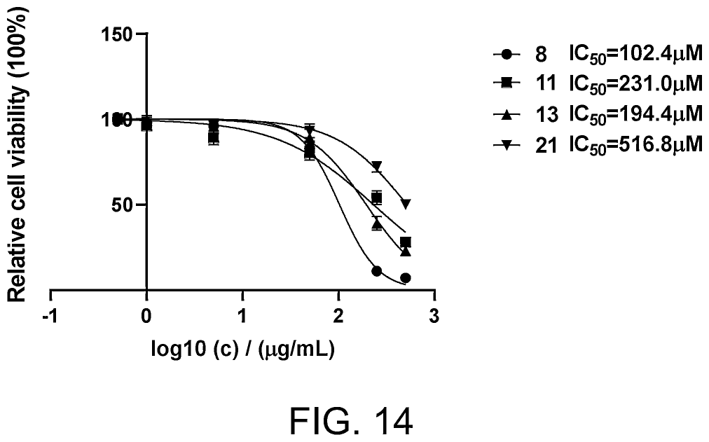

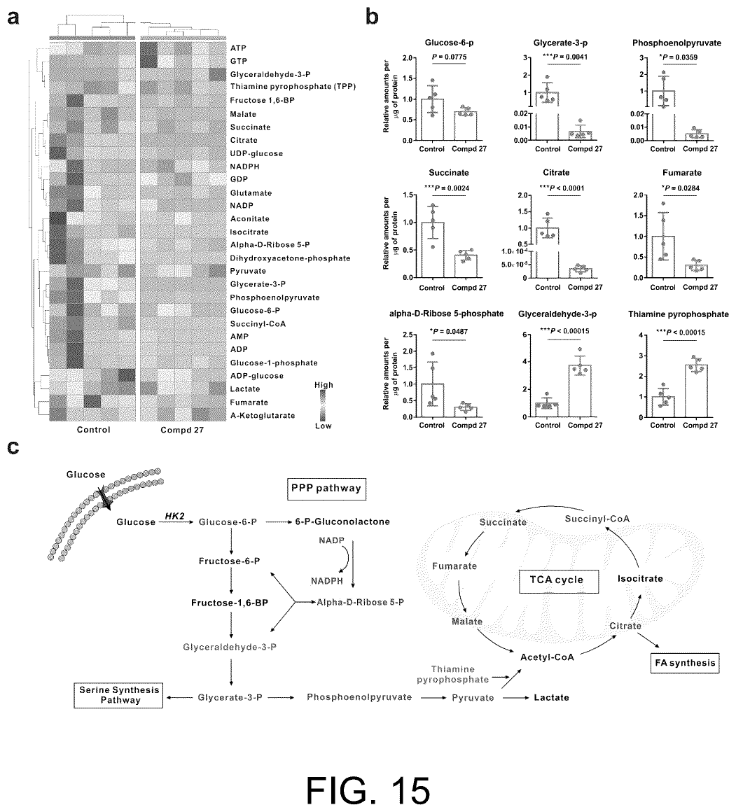

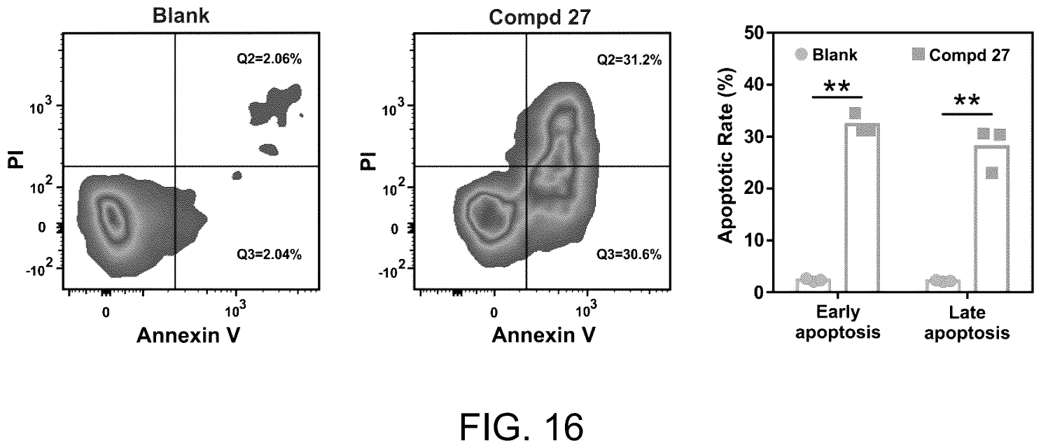

FIG. 8-2 shows pharmacokinetic data of compound 27;

FIG. 9 is an MSI image of a drug and a metabolite (compound 27: m/z 499, lactic acid: m/z 113) in a brain tissue section;

In FIG. 10, a shows a U87MG subcutaneous tumor growth curve of a nude mouse after elimination of a primary tumor by means of various treatments, an error bar represents a mean+/−s.d, (n=6), b is a survival rate (n=6 for each group) of a mouse with a subcutaneous U87MG tumor after various treatments, and c shows a representative bioluminescence image;

FIG. 11 shows an antiproliferative activity of compound 27 against a cranial nerve glioma cell U87;

In FIG. 12, a is a schematic diagram of a combination structure of a comp-27-HK compound subjected to a Glide XP docking simulation prediction, and b is a two-dimensional schematic diagram of a combination mode of a composite 27-HK complex having hydrogen bonds and strong hydrophobic interaction;

FIG. 13 shows difference in an interaction score (Eintcompd 27-Eint 3-Br) of compound 27 and 3-Br on each residue;

FIG. 14 is an antiproliferative activity test diagram of compound 8, compound 11, compound 13, and compound 21 against a cranial nerve glioma cell U87;

FIG. 15 shows normalization of a glioma U87 cell reprogramming metabolic pathway after compound 27 treatment; and

FIG. 16 shows a flow cytometry analysis of U87 cell apoptosis (Annexin V-FITC/PI staining) induced by compd 27.

### DETAILED DESCRIPTION OF THE EMBODIMENTS

In order to further illustrate the present invention, an application of a small-molecule compound in preparation of an antitumor drug provided by the present invention is described in detail below with reference to the examples, which cannot be construed as a limitation to the protection scope of the present invention.

### Example 1

Sources of 38 Candidate Inhibitors:
1. compound library acquisition (240 thousand compounds, acquisition being achieved by performing screening by using a LigPrep module of Schrödinger in a Specs compound library); and
2. docking simulation (precision mode: standard precision (SP) and extra precision (XP)) between the compound and hexokinase (HK)2 was made by Glide of Schrödinger (a protonation state parameter is pH=7.0+/−2.0, a scale factor of the Van der Waals radius was set to be 1.0, and a maximum partial atomic charge was set to be 0.25).

Specific docking process: all the compounds were structurally docked to HK2, and binding affinity was scored and sorted by using a Glide-SP docking mode. Then, the 50000 compounds ranking top were scored by using a Glide XP docking mode. Next, the absorption, distribution, metabolism, excretion, toxicity (ADMET) properties of the selected compounds were predicted by using an ACD/ADME software package, and the 1000 compounds ranking top in Glide XP docking were filtered to remove those compounds which did not conform to the following rules: (1) log P/log D (pH=7.0)<5.5, (2) violating the Lipinski Rule of Five and being <2, (3) violating a drug similarity rule of Opera and being <3, and (4) functional groups having no toxicity, reactivity or other bad portions defined by the REOS rule. Then, the remaining molecules were clustered by using a Find Diversity Molecule module in Discovery Studio 2.5 according to a Tanimoto distance calculated by FCFP_4 fingerprints. Finally, 40 hit compounds with the lowest docking scores were purchased from the Specs library.

Structural formulas of the 40 compounds are as follows:

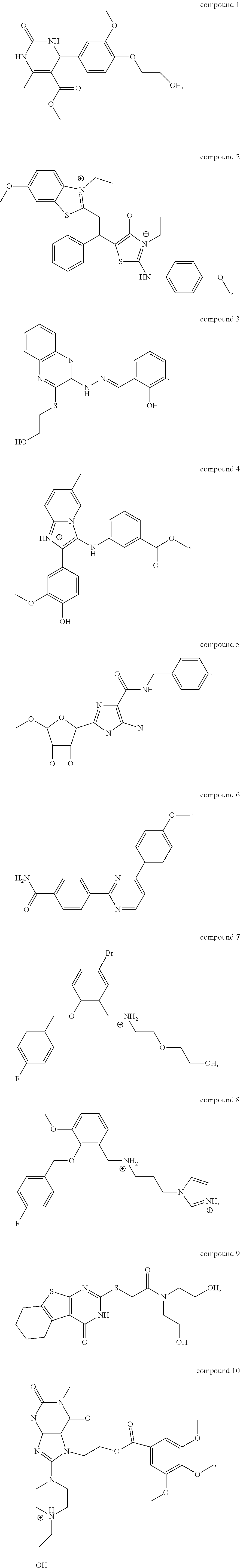

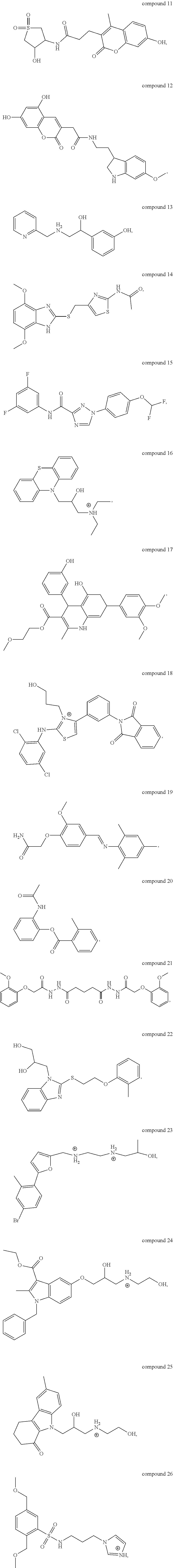

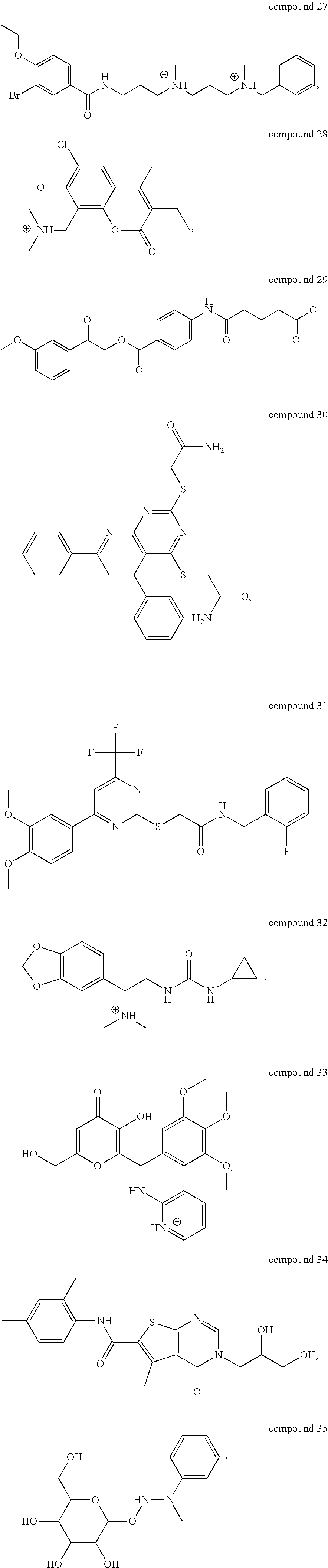

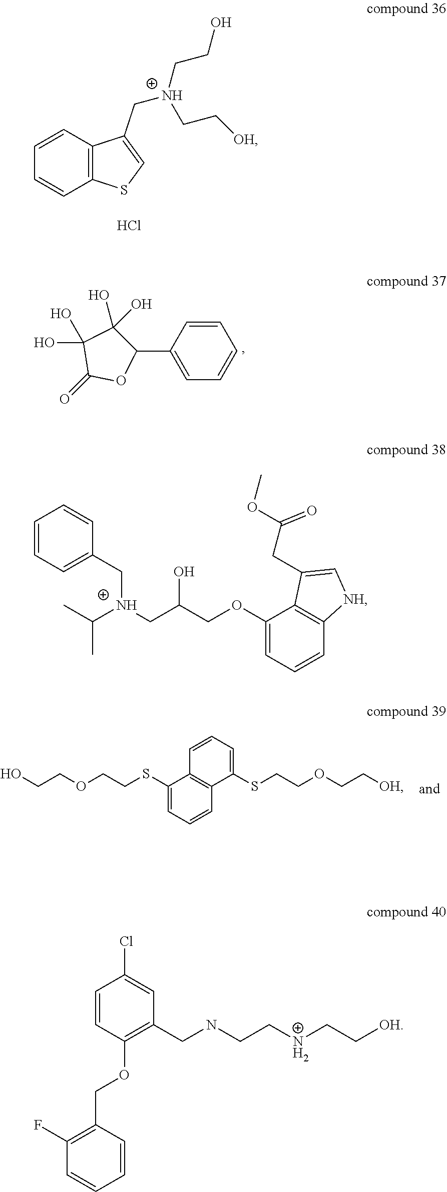 [SMILES: OCC[NH+](CCO)Cc1csc2ccccc12 ; Cl ; O=C1OC(c2ccccc2)C(O)(O)C1(O)O ; COC(=O)Cc1c[nH]c2cccc(OCC(O)C[NH+](Cc3ccccc3)C(C)C)c12 ; OCCOCCSc1cccc2c(SCCOCCO)cccc12 ; OCC[NH2+]CCNCc1cc(Cl)ccc1OCc1ccccc1F]

In the above-mentioned compounds, compound 2, compound 33 and compound 34 were excluded due to poor water solubility, and the remaining 37 compounds were used as candidate inhibitors to continue testing. An HK2 activity test was performed in a 50 μL reaction buffer solution, the buffer solution was composed of 10 mM of glucose, 1.2 mM of adenine nucleoside triphosphate (ATP), 2.5 μL of HK2 (0.1 mg/mL), 25 mM of a Tris-HCl buffer solution and 5 mM of MgCl₂, and a pH value was 7.5. 37 candidate inhibitors with different concentrations were added at a volume ratio of the reaction buffer solution to the candidate inhibitor of 1:1, the concentration of the candidate inhibitor was selected to be 1 μmol/L, 10 μmol/L, 100 μmol/L and 1 mmol/L according to experimental requirements, and the reaction mixture was incubated at 37° C. for 60 minutes on a heating oscillation reactor. The reaction was stopped by adding trifluoroacetic acid (TFA) as a terminator until the final concentration was 2% (v/v). Three times of parallel experiments were performed on each small-molecule inhibitor. After an equal volume of 0.5 mM glucose-1-¹³C was added to the solution after the reaction was terminated, 1 μL was taken as a sample to be analyzed for mass spectrometry.

Preparation Step A) of GDs: Synthesis of Graphite Dots

The initial graphite dots were obtained through an electrochemical corrosion method. Firstly, two graphite rods (99.99%, Alfa Aesar Co. Ltd) were inserted in parallel into deionized water, one as an anode and the other as a cathode, and moreover, a static voltage between the two electrodes were guaranteed to be 30 V. The whole electrolysis process lasted for two weeks, and high intensity magnetic stirring was maintained continuously, and then the initial graphite quantum dots were produced. However, a graphite quantum dot solution at this time was also mixed with large graphite particles, such that the solution needed to filtered and centrifuged at a high speed (22000 rpm, 30 min) to obtain graphite dots with uniform particle size and quite good water solubility.

Step B): Preparation of Reduced Graphite Dots

Sodium borohydride reduction was a mild process which occurred at a room temperature and only selectively reduced carbonyl groups (C═O) and epoxy groups. The specific steps were as follows: 300 mg of the obtained graphite dots were weighed and dissolved in 300 mL of water, and then an appropriate amount of sodium borohydride (concentration of 150 mM) was added. Magnetic stirring was performed for reaction for 6 hours at a room temperature. Then, dialysis was performed for 3 days by using dialysis bags to obtain reduced graphite dots (GDs). Finally, the product was finally dried in an oven at 60° C. for 12 hours.

GDs were dispersed in water at a concentration of 1 mg/mL as a matrix solution to be used. A sample was prepared by using a quick-drying method: firstly, 1 μL of matrix solution was dropped onto a target plate, and after natural drying at a room temperature in a high magnetic field of 10000 V electric field, 1 μL of sample to be analyzed was dropped onto a surface of the dried matrix, and after the sample was naturally dried, mass spectrometry was directly performed:

An instrument in the MALDI-MS was a Bruker Ultraflex III TOF/TOF mass spectrometer, which was mainly in a reflection positive and negative ion mode. Nd of 355 nm for instrument parameter: YAG laser had laser energy of 30% corresponding to 57 μJ per pulse (laser pulse duration: 3 ns), a laser spot size was about 50-100 μm, each sample was repeatedly tested four times, and 3000 laser spots were accumulated in each mass spectrum. All samples were measured under the same instrumental conditions.

After mass spectrometry, screening was performed according to the following screening conditions:
a concentration of the candidate inhibitor aqueous solution was 20 μmol/L, an inhibition rate of hexokinase 2 activity was ≥25%, and

an inhibition rate=(1−B/A)×100%, where
A was: a glucose concentration in a pre-reaction solution without the inhibitor-a glucose concentration in a post-reaction solution without the inhibitor,
B was: a glucose concentration in a pre-reaction solution with the inhibitor-a glucose concentration in a post-reaction solution with the inhibitor, and
a glucose concentration in the reaction solution=(mass spectrum signal strength of [glucose+Na]+/mass spectrum signal strength of [glucose-1-¹³C+Na]+)×known concentration of glucose-1-¹³C.

Compound 8 (having the structure of formula 1), compound 11 (having the structure of formula 2), compound 13 (having the structure of formula 3), compound 21 (having the structure of formula 4), and compound 27 (having the structure of formula 5) were obtained by means of screening.

FIG. 1 is a schematic diagram of a GLMSD platform for high-throughput screening of a hexokinase 2 (HK2) candidate inhibitor. With 102 concentrations×3 parallel test experiments×6 parallel samples, each sample was tested for 10 s. Regarding the ultra-fast small-molecule drug screening detection speed, 1836 samples were analyzed within 5.1 hours. The platform was docked to pharmacodynamic analysis of the small-molecule drugs, and there was no need for transfer to any other testing platform.

FIG. 2 shows inhibition rates of 38 candidate inhibitors against HK2 at four different concentrations. It can be seen from FIG. 2 that compared with a commonly used HK2 colorimetric screening method, inhibition rates of the compounds obtained through the method provided by the present application and the colorimetric method are similar, indicating that the GLMSD testing results provided by the present application are accurate. In the figure, from red to purple, the inhibition efficiency is from strong to weak, and gray indicates that a testing kit fails to perform testing. In addition, 3-bromopyruvic acid (3-BP, a commonly used HK2 inhibitor) was selected to be used as a positive control, and five new small molecules (a small-molecule concentration of the inhibitor being 20 μM), namely compound 8 (corresponding to formula 1 and having an inhibition rate of 44%), compound 11 (corresponding to formula 2 and having an inhibition rate of 29%), compound 13 (corresponding to formula 3 and having an inhibition rate of 42%), compound 21 (corresponding to formula 4 and having an inhibition rate of 25%), and compound 27 (corresponding to formula 5 and having an inhibition rate of 31%) were obtained by means of screening and showed good inhibitory capacity against the HK activity.

The hexokinase colorimetric method is not as extensive as the GLMSD platform, because the colorimetric method is to monitor change of ultraviolet absorption at 340 nm to determine the enzyme activity, but many small molecules have strong ultraviolet absorption in this range, such as compound 3, compound 4, compound 6, compound 9, compound 11, compound 23, compound 25, compound 26, compound 28 and compound 39, and absorption peaks of these compounds change shapes and positions of absorption peaks to be measured, which disturb testing results. Due to this limitation, 10 compounds failed to be successfully tested for inhibition during the conventional colorimetric method assay process, compound 11 therein showed a good inhibition effect in the GLMSD platform assay, and therefore, the GLMSD platform allows the assessment of inhibition efficiency for all the small molecules, independent of ultraviolet absorption peaks.

In FIG. 3, a is a structural formula of compound 8, b shows inhibition curves of HK2 from compound 8 by means of a colorimetric kit and GLMSD, and c shows original data of an HK2 activity of compound 8 at four concentrations tested by a GLMSD platform.

In FIG. 4, a is a structural formula of compound 11, b shows inhibition curves of HK2 from compound 11 by means of a colorimetric kit and GLMSD, and c shows original data of an HK2 activity of compound 11 at four concentrations tested by a GLMSD platform.

In FIG. 5, a is a structural formula of compound 13, b shows inhibition curves of HK2 from compound 13 by means of a colorimetric kit and GLMSD, and c shows original data of an HK2 activity of compound 11 at four concentrations tested by a GLMSD platform.

In FIG. 6, a is a structural formula of compound 21, b shows inhibition curves of HK2 from compound 21 by means of a colorimetric kit and GLMSD, and c shows original data of an HK2 activity of compound 21 at four concentrations tested by a GLMSD platform.

In FIG. 7, a is a structural formula of compound 27, b shows inhibition curves of HK2 from compound 27 by means of a colorimetric kit and GLMSD, and c shows original data of an HK2 activity of compound 11 at four concentrations tested by a GLMSD platform.

Corresponding inhibitory effects of five compounds, namely compound 8, compound 11, compound 13, compound 21 and compound 27 can be seen from FIGS. 3-7, and all these compounds have good inhibitory effects on the HK2.

FIG. 8-1 shows pharmacokinetic data of Trimetazidine (TMZ), where a shows an average concentration-time curve of TMZ in plasma after TMZ is injected into an abdominal cavity of a mouse, b shows median analysis performed on a TMZ pharmacokinetic curve of each mouse, and c shows a pharmacokinetic curve determined by means of GLMSD for multiple administration. It can be seen from FIG. 8-1 that the GLMSD platform can specifically acquire the pharmacokinetic data of small-molecule compound 27. Competitive advantages of GLMSD in pharmacokinetic research: (1) a complete drug blood concentration curve of each individual may be tracked; and (2) the total number of mice used in the test is reduced. Since the blood sample consumption of the GLMSD method is low (10 μL of blood per time point), one mouse can be used for completing the entire pharmacokinetic curve (blood sampling at 11 time points within 24 hours, and the minimum interval being 0.25 hour) of a drug. The average TMZ pharmacokinetic curve (d in FIG. 8-1) of eight mice (n=8) provides detailed values of Cₘₐₓ (33.66 μg/mL), Tₘₐₓ (0.5 h), T_{1/2} (1.72 h) and AUC_{0-24 h} (247.05 μg/mL·h) which are similar to previous literature research data, proving that the GLMSD method can complete pharmacokinetic research. In FIG. 8-1, b shows an average plasma concentration of 8 mice taking TMZ every day for 5 continuous days.

After the superiority of the GLMSD method in the pharmacokinetic testing is verified, we further use the method to characterize a pharmacokinetic curve of compound 27, which is shown in FIG. 8-2, and FIG. 8-2 shows pharmacokinetic data of compound 27. 8 mice were administered once (at a single dose of compound 27 of 40 mg/kg), and results of the plasma pharmacokinetic (PK) value were shown in b in FIG. 8-2, including Cₘₐₓ (14.65 μg/mL), Tₘₐₓ (2 h), T_{1/2} (4.12 h) and AUC_{0-24 h} (64.36 μg/mL·h). Based on the advantage that the complete PK curve can be tested by means of the GLMSD, we observe that 3 (#2, #3 and #4) in 8 mice have the characteristic re-absorption peak of this small-molecule inhibitor at the time point of 8 h, and such a characteristic re-absorption peak hardly exists in other 5 mice. This significant phenomenon is related to drug uptake differences between individuals, prompting that compound 27 may re-enter blood circulation by means of a secondary absorption pathway. In the mode of continuous multiple administration (c in FIG. 8-2), pharmacokinetic data of compound 27 (40 mg/kg) was collected from each mouse every day (1, 3, 6, and 8 hours after last administration) for 5 days. For a single dose (a in FIG. 8-2) or a continuous multi-dose (d in FIG. 8-2) administration manner, a limitation of a conventional LC-MS technology lies in obtaining a single data point of pharmacokinetics of a drug by sacrificing one mouse, whereas GLMSD can break through this limitation by providing a complete pharmacokinetic curve based on a single mouse.

FIG. 9 is an MSI image of a drug and a metabolite (compound 27: m/z 499, lactic acid: m/z 113) in a brain tissue section, where an optical micrograph of a serial section of H&E staining is used as a reference. The brain glioma and the lateral ventricle are represented by dashed lines, a color bar encodes signal strength of three small molecules in multispectral scan imaging (MSI), and the resolution is 10 μm. FIG. 9 shows that compound 27 has the capacity of penetrating a blood-brain barrier. A superimposed image of 100-1000 small molecules (left first figure) clearly shows a longitudinal section structure of the brain (U87 cell transplantation tumor being located at the right frontal lobe), which is consistent with the adjacent longitudinal serial tissue section (left second figure) dyed by H&E. The left third figure shows that compound 27 mainly exists in contour edges of the corpus callosum and the hippocampus. The left fourth figure shows that a content of lactic acid (m/z 113.35) around the glioma implant site is significantly higher than that of other brain tissues. The glioma portion is enriched with compound 27, which indicates that this small molecule has good permeability and can break through the blood-brain barrier.

In FIG. 10, a shows a U87MG subcutaneous tumor growth curve of a nude mouse after elimination of a primary tumor by means of various treatments, an error bar represents a mean+/−s.d, (n=6), b is a survival rate (n=6 for each group) of a mouse with a subcutaneous U87MG tumor after various treatments, and c shows a representative bioluminescence image, thereby tracking cancer growth in situ U87-luciferase mice after various treatments. It can be seen from FIG. 10 that compound 27 can inhibit growth of solid tumors. In all different experimental groups (a and b in FIG. 10), the overall survival (OS) of mice treated with TMZ and compound 27 is the best. The median survival time (58 days) of TMZ+compound 27 is significantly longer than other therapeutic groups: PBS (15 days), 3-BP (17 days), compound 27 (30 days), TMZ (35 days) and TMZ+3-BP (35 days). These experimental results show that as the HK2 inhibitor, compound 27 is combined with TMZ and other chemotherapeutic drugs for cancer treatment, which have good drug adaptability. Compared with PBS (c in FIG. 10), the single drug treatment in which TMZ or compound 27 is used alone delays the growth of glioma. However, mouse death caused by glioma is reduced only 20 days before treatment. After this period of time, the survival rates of the in-situ U87 heterogeneous tumor of the two single drug groups decreases rapidly due to malignant glioma. In sharp contrast, the treatment group in which TMZ is combined with compound 27 can inhibit the U87 glioma of bioluminescence and significantly increase the survival rate.

FIG. 11 shows an antiproliferative activity of compound 27 against a cranial nerve glioma cell U87. It can be seen from FIG. 11 that the compound 27 can inhibit growth of U87, and when a concentration is 11.31 μM, half of the tumor cells can be killed.

In FIG. 12, a is a schematic diagram of a combination structure of a comp-27-HK compound subjected to a Glide XP docking simulation prediction, and b is a two-dimensional schematic diagram of a combination mode of a composite 27-HK complex having hydrogen bonds and strong hydrophobic interaction. Active site residues Phe156, His159, Ser155, Cys158, Asn235, Asp209, Glu294, Ile229, Asn208 and Glu260 of the HK2 are all combined with compound 27. Bonding of Asp209, Ile229 and Glu260 on the HK2 to compound 27 is the most critical. Glu260 forms a hydrogen bond with a nitrogen atom of the amide, and the residue Asp209 forms two hydrogen bonds. The hydrophobic contact (aromatic-H interaction) between Ile229 and the benzene ring of compound 27 also facilitates binding. FIG. 12 shows that small-molecule compound 27 and the HK have many bonding sites.

FIG. 13 shows difference in an interaction score (Eintcompd 27-Eint 3-Br) of compound 27 and 3-Br on each residue, where significant residues (amino acid residues constituting protein by means of dehydration) are highlighted. The total interaction energy, Van der Waals force and hydrogen bond score between the ligand and the protein residue are calculated by means of the Glide module in Schrödinger, and by means of theoretical calculation, the binding force between small-molecule compound 27 and the HK is strong than that between the common HK inhibitor 3-Br and the HK. Comparison of a binding mechanism between compound 27 and 3-BP is presented in the figure, showing that binding sites of compound 27 and the HK2 are more, which may be a reason why compound 27 has a better inhibitory effect on the HK2 activity.

FIG. 14 is an antiproliferative activity test diagram of compound 8, compound 11, compound 13, and compound 21 against a cranial nerve glioma cell U87. It can be seen from FIG. 14 that all the above 4 small-molecule compounds obtained by means of screening on the GLMSD platform have inhibitory effects on U87.

### Example 2 Targeted Metabonomics Analysis

1. Metabolite Extraction

1×10⁷ cells (a sample cell number) were collected, and the cell supernatant was removed by means of centrifugation (1000×g, 1 minute) after quenching with a newly prepared quencher. The cells were resuspended with 100 μL of ultrapure water and mixed well. After 800 μL of cold methanol:acetonitrile (1:1, v/v) was added, the uniformly mixed cell samples were subjected to ultrasonic treatment in an ice bath for 30 minutes. The mixture was stored at −20° C. for 1 h to precipitate the protein. After centrifugation at 4° C. (14000×g, 20 minutes), the supernatant was collected, lyophilized, stored at −80° C., and then analyzed by means of GC-MS.

2. Sample Testing

The lyophilized samples were resuspended in a solution (acetonitrile:water, 1:1, v/v, 100 μL) and then centrifuged at 4° C. (14000×g, 10 minutes). The supernatant (100 μL) was collected and diluted with an acetonitrile solution (100 μL), and a GS-MS sample was loaded at 4° C. by using an Agilent 1290 Infinity liquid chromatography (LC) system (Agilent Technologies, Beijing, China) and a 5500 QTRAP mass spectrometer (Toronto of Canada, AB Sciex). At ACQUITY-UPLC-BEH column (1.7 μm, 2.1 mm×150 mm, Waters Technology (Shanghai) Co., Ltd.), chromatographic separation was performed at a flow rate of 300 μL/min, where solvent A (mobile phase) was a 15 mM ammonium acetate aqueous solution, and solvent B was acetonitrile. Chromatographic conditions of gradient elution were reduced from 90% B to 40% B, and were increased sharply from 40% B to 90% B after 18 minutes, a volume ratio of 90% B was kept for 4.9 minutes after 0.1 minute, and the duration of the whole process was 23 minutes. An equal volume of compounds was extracted from 32 actual samples so as to monitor the stability of the system, thereby being used for processing a quality control (QC) sample and put into a real sample. Mass spectrum experiments were performed in a negative ion and multi-reaction monitoring mode, and specific parameters were as follows: a source temperature of 450° C., an atomizer gas (GS1): 45, an auxiliary gas (GS2): 45, a curtain gas (CUR): 30, and an ion space voltage floating (ISVF): −4500 V.

3. Data Processing

Standard GC-MS data was processed by using analysis software (AB Sciex, Toronto, Canada), which included converting the original mass spectrum data to data containing m/z, measuring the corresponding ion intensity and retention time, and subsequent statistical analysis. Peak testing and calibration of all the samples were compared to their chemical standards (Sigma-Aldrich). The data matrix was uploaded to MetaboAnalyst 5.0 for principal component analysis (PCA) and hierarchical clustering analysis (HCA).

4. Flow Cytometry Experiment

In a dulbecco's modified eagle medium (DMEM), 5×10⁵ U87 cells were inoculated into a 24-hole plate. After culturing for 24 hours, compound 27 (final concentration 20 μM) was directly added to a cell growth medium, incubated at 37° C. for 24 hours, briefly digested with trypsin, washed twice with cold PBS, centrifuged (2000 rpm, 5 min) and washed twice with cold PBS after a supernatant culture medium was discarded. The cells (1×10⁵ cells/mL) were resuspended with a 400 μL of buffer solution (1×). 5 μL of Annexin V-FITC was added to the cell suspension and cultured at 4° C. for 15 minutes. Then, the cells were gently mixed with another dye propidium iodide (PI) (10 μL). The cells were collected for flow cytometry analysis. Data analysis was performed by using a CFLow Plus (Accuri Cytometers).

FIG. 15 shows normalization of a glioma U87 cell reprogramming metabolic pathway after compound 27 treatment, where a shows a relative difference heat map of U87 cell aggregation metabolites treated by compound 27 and a blank control, n=5, and b shows quantitative analysis of the metabonomics change of U87 cells in glycolysis and a trichloroacetic acid (TCA) cycle. Statistical significance was tested by using t. *: P<0.05, **: P<0.01, and ***: P<0.005. C shows visualization of differential glycolysis metabolite expression after compound 27 inhibition. The applicant observes that the change in a reprogramming metabolic pathway in U87 glioma cells treated with the HK2 candidate inhibitor (compound 27) has significantly different metabolite cluster performance compared to the control group processed by PBS (a in FIG. 15). The hexokinase (HK2 in cancer cells), which is the first one and serves as a rate-limiting enzyme in a glycolysis pathway was evaluated. The metabonomics analysis shows that the concentration of the hexokinase direct product glucose-6-phosphate was lower than that of the control group. The decrease in glycerol-3-phosphate, phosphoenolpyruvic acid and pyruvic acid indicates that compound 27 inhibits glycolysis of U87 cells, thereby reducing the basic energy reserve of tumor growth. Due to inhibition of compound 27 on the HK2, an abnormal pentose phosphate pathway (PPP) of U87 is also suppressed. The precursor (α-D-ribose-5-phosphate) and the key cofactor (nicotinamide adenine dinucleotide phosphate, NADPH) synthesized by the ribonucleotides are both reduced, thereby limiting ribose supply by PPP DNA synthesis required for tumor proliferation. In addition, previous reports have shown that glycerate-3-phosphate is also an important metabolic intermediate in a serine synthesis pathway (SSP). After treatment with compound 27, U87 cell glyceride-3-phosphate is significantly reduced by nearly two orders of magnitude (P<0.005), showing defects of U87 cells in denovo synthesis of serine and glycine. Blocking (P<0.0001) generated by citrate in the TCA cycle further inhibits the synthesis of FA in U87 cells, leading to insufficient lipid, and failing to satisfy needs of the tumor cell activity. In addition, after the applicant finds that after treatment with compound 27, the concentration of thiamine pyrophosphate (TPP) is increased by nearly 2.5 times, and TPP is a key coenzyme for converting pyruvic acid into acetyl coenzyme a by adjusting the activity of pyruvate dehydrogenase (PDH), which promotes apoptosis of U87 cells by reducing the glucose metabolic flux of TCA. Glyceraldehyde-3-phosphate and α-D-ribose-5-phosphate are intermediate metabolites in glycolysis pathways, and reversible changes can occur. Experiments show that compound 27 normalizes the pentose phosphate pathway, reduces the generation of α-D-ribose-5-phosphate while accumulating glyceraldehyde-3-phosphate (approximately 3.5 times higher than that of the control group processed by PBS). In summary, these data reveal significant change in U87 cell metabolic pathway normalization induced by means of compound 27.

In order to further confirm the occurrence of apoptosis, the applicant tested the exposed phosphatidylserine by using an annexin V/propidium iodide double staining method, and observed a cell membrane damage phenomenon. With reference to FIG. 16, FIG. 16 shows a flow cytometry analysis of U87 cell apoptosis (Annexin V-FITC/PI staining) induced by compd 27, and two main cell populations are observed by the flow cytometer: compared with the control group, early (Annexin+/PI−) and late (Annexin+/PI+) apoptotic cells of compound 27 treatment group account for 31.2% and 30.6% respectively, whereas in the blank control group, the proportions of U87 damaged cells are only 2.06% and 2.04%. These results indicate that the glycolysis inhibition pathway of compound 27 participates in apoptosis of glioma cells.

It can be seen from the above examples that the present invention provides the method for screening the hexokinase 2 inhibitor. The method includes the following steps: the candidate inhibitor and the buffer solution were mixed and incubated, and a reaction was terminated to obtain a post-reaction solution, where the buffer solution included the hexokinase 2 and the glucose; the post-reaction solution was uniformly mixed with the equal volume of glucose-1-¹³C to obtain the object to be analyzed; and the object to be analyzed was dropwise added to the surface of the graphite structure type nanomaterial matrix and subjected to the MALDI-MS testing after drying, and screening was performed to obtain the hexokinase 2 inhibitor. According to the method, screening and testing are performed at an ultra-rapid speed (1836 samples are analyzed within 5.1 hours) by taking the graphite structure type nanomaterial as the matrix in combination with the MALDI-MS testing. A series of small-molecule drugs with high brain tumor growth activity inhibitory properties are obtained, are docked to pharmacodynamic analysis of the small-molecule drugs, and do not need to be transferred to other testing platforms.

The above mentioned description are merely the preferred embodiments of the present invention, it should be pointed out that those of ordinary skill in the art can also make some improvements and modifications without departing from the principle of the present invention, and these improvements and modifications should also fall within the protection scope of the present invention.

What is claimed is:

1. An application of a small-molecule compound in preparation of an antitumor drug, wherein the small-molecule compound has a structure of formula 1 to formula 45:

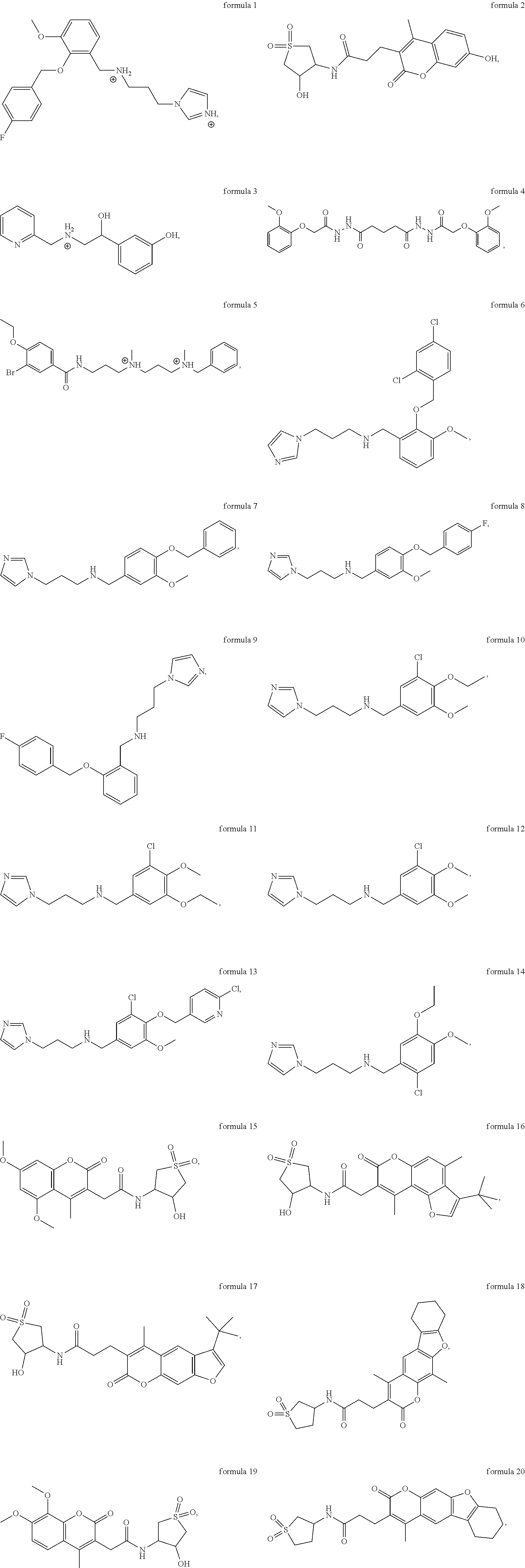

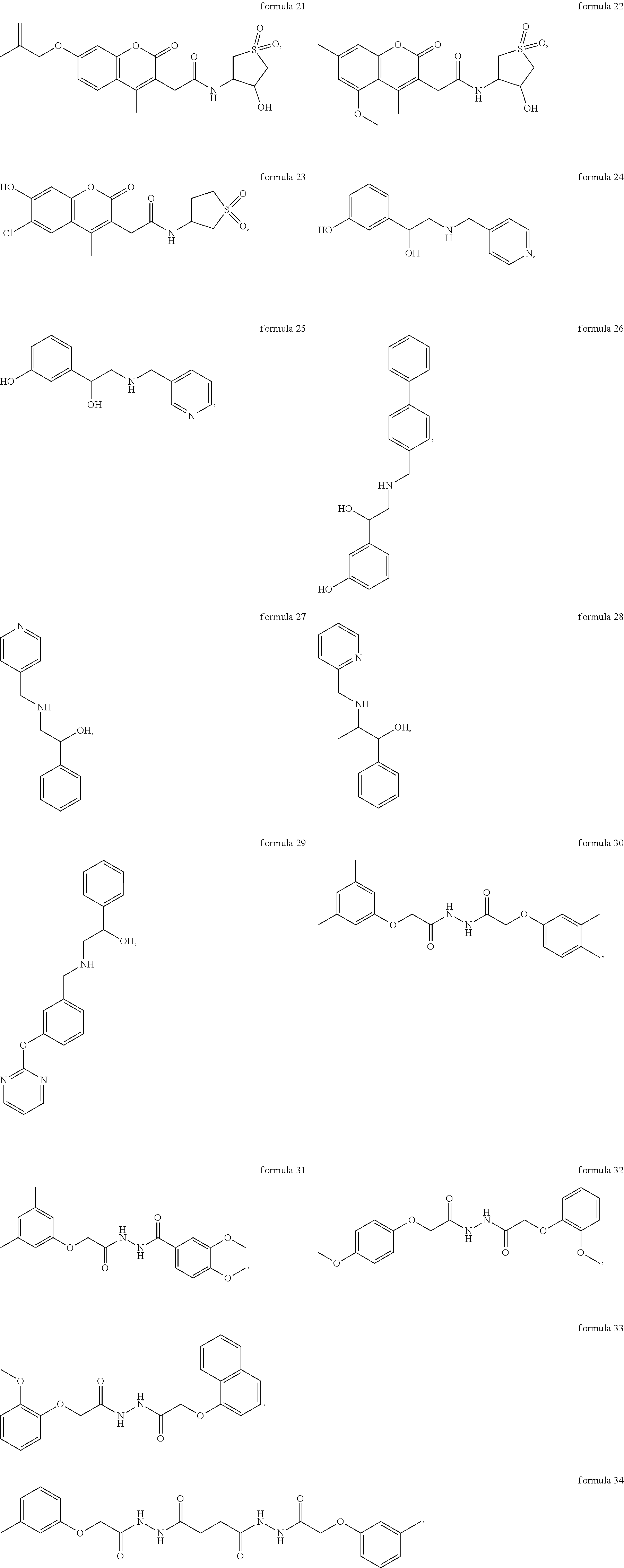

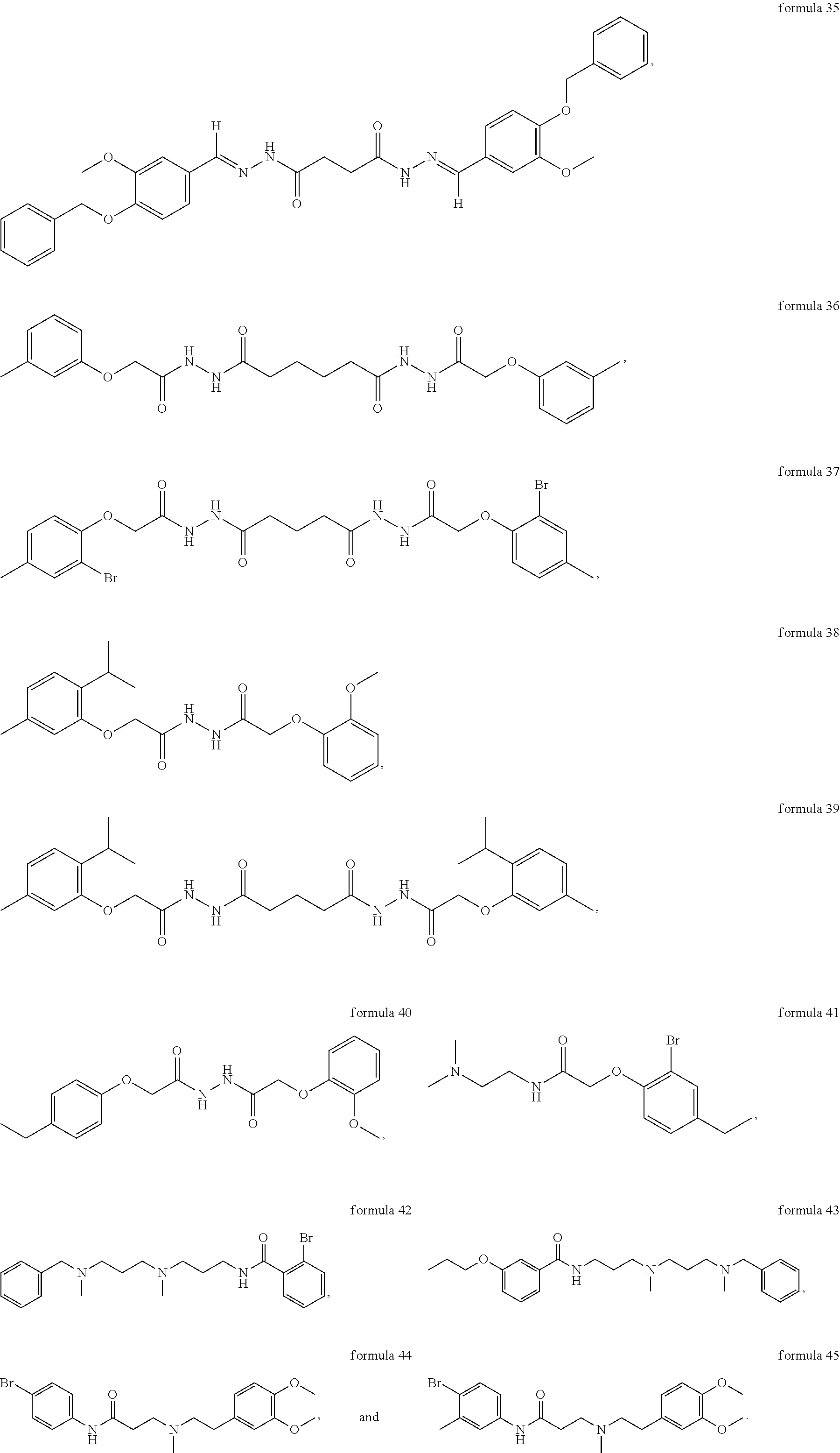

2. The application according to claim 1, wherein the small-molecule compound has an inhibitory effect on a key protease in glycolysis abnormality caused by metabolic reprogramming of tumor cells, and is used alone as a pharmaceutical molecule or used in combination with other known drugs to kill the tumor cells.

3. The application according to claim 1, wherein the small-molecule compound can hinder the capacity of the tumor cell of repairing damaged deoxyribonucleic acid (DNA) during chemotherapy by inhibiting an abnormal metabolic pathway of the tumor cells; and
the small-molecule compound can break through restriction of a blood-brain barrier, and is captured and enriched at a tumor site where a drug is usually difficult to reach.

4. The application according to claim 1, wherein in the case of single or consecutive injection of the small-molecule compound, quantitative mass spectrometry analysis of drug molecules is performed on a sample with a blood taking amount of less than 10 microliters, such that data acquisition of pharmacokinetics (blood drug concentration-time curve) can be completed by means of a single model animal.
